(19) United States
(12) Patent Application Publication
(10) Pub. No.: US 2007/0142781 A1
(43) Pub. Date: Jun. 21, 2007

(54) MICROINJECTOR CHIP

(76) Inventor: Chauncey B. Sayre, Irvine, CA (US)

Correspondence Address: KIRKPATRICK & LOCKHART PRESTON GATES ELLIS LLP, 1900 MAIN STREET, SUITE 600, IRVINE, CA, 92614-7319, US

(21) Appl. No.: 11/614,899

(22) Filed: Dec. 21, 2006

Related U.S. Application Data
(60) Provisional application No. 60/753,208, filed on Dec. 21, 2005.

(51) Int. Cl. A61M 5/00

(52) U.S. Cl. 604/173; 604/258

(57) ABSTRACT

A microinjector chip, and associated methods, for microinjecting a plurality of cells with injection materials is provided wherein the microinjector chip comprises a plurality of projections protruding in parallel from and perpendicular to a top surface of the microinjector chip.

### CROSS REFERENCE TO RELATED APPLICATIONS

The present application claims priority under 37 U.S.C. §119(e) to U.S. Provisional Patent Application No. 60/753,208 filed Dec. 21, 2005, the entire contents of which is incorporated by reference herein.

### FIELD OF THE INVENTION

The present invention provides devices and methods for delivering injection materials such as organic or inorganic molecules into cells by microinjecting them directing into the cells. More specifically the present invention provides a microinjector chip device having projections wherein molecules are coated or dried onto the projections and are used to deliver the injection materials into target cells.

### BACKGROUND OF THE INVENTION

Direct injection of materials into cells is currently only a viable technique in a limited number of fields, for example in vitro fertilization, and currently is carried out manually and individually on each cell. It requires a high level of skill and an experienced operator can only inject in the order of one cell per minute. There are many other fields that would benefit from cell injection of macromolecules, genes, chromosomes, organelles, or any other injection material desired to be injected into a cell were it possible to achieve this effect on a large numbers of cells.

Currently available methodology for introducing molecules into cells include injecting materials directly into single cells (microinjection) or groups of cells (biolistic approaches) or making the cells more permeable so as to allow uptake of desired molecules from a surrounding medium (micropricking, transfection, electroporation).

In single cell microinjection, cells are suspended in solution and each cell is individually injected by fixing a cell into position by the operator “sucking” the cell onto the end of a narrow pipette. While watching the cell positioning through a microscope the operator then inserts a needle into the cell. Once the injection is made the needle is retracted manually and the cell is released. Variations of this basic manual technique are available such as, for example, for injecting cells which are attached to a dish as a monolayer. The cost of injecting a small number of cells is expensive and therefore single cell microinjection is not a technique used widely.

In a variant of single cell microinjection termed “micropricking”, a cell wall is ruptured with a needle and the surrounding medium, containing the injection material, is allowed to diffuse into the cell through the break in the cell wall. However, like single cell microinjection, this procedure requires a high degree of manipulative skill by the operator and is very time consuming.

Another methodology for inserting injection materials into cells, most often used for the introduction of nucleic acids, such as gene constructs, is the “biolistics” approach wherein high density metallic particles, usually of tungsten or gold, are coated with the nucleic acids and are propelled by gas release at a target cell culture. This approach does not have the precision of microinjection or micropricking but takes the “shotgun” approach which exposes a large number of cells to the injection material with the expectation that many of the cells will take up the injection material. While this method has the potential to reach large numbers of cells relatively easily, it requires expensive equipment and the force of the gas release may harm the target cells.

Related mechanisms such as transfection or electroporation are also used wherein the cells are made porous and encouraged to take up injection materials, such as nucleic acids and gene constructs, from the surrounding medium.

All of the methods discussed supra have one common drawback. In each of these methods, the injection material is suspended in a liquid, and the amount of injection material provided to each cell is dependent on the amount of material which can be suspended in the liquid and the volume of liquid that can be injected into the cell. In order to increase the amount of injection material provided to a cell, it is advantageous to limit the amount of liquid. Therefore methods that allow injection of material in little or no liquid are desired.

Further, none of the described methods provide scientists with a means to directly inject highly concentrated molecules directly into large numbers of cells efficiently. Therefore, an unmet need also exists for devices and methods in which large numbers of cells can be microinjected with a desired concentration of molecules with minimal operator involvement.

### SUMMARY OF THE INVENTION

The present invention provides a microinjector chip device for the rapid injection of a large number of cells with minimal operator involvement and minimal dilution of the target molecule with aqueous solutions. The microinjector chip device has a first surface and a second surface and a plurality of projections extending from the first surface about perpendicular to the surface. Injection materials are coated, or deposited, onto the projections allowing for the substantially liquid-free transfer of the injection materials into the cells. The microinjector chip device pierces the target cells and the injection material coated on the projections are deposited within the cells. Methods of making the microinjector chip devices, coating them with injection materials and delivering the injection materials to target cells are also provided.

In one embodiment of the present invention, a microinjector chip for delivery of injection materials to a plurality of cells is provided comprising: a microinjector chip having a first surface and a second surface; and a plurality of projections protruding from the first surface wherein the injection materials are coated onto at least a subset of the projections. In another embodiment, the plurality of projections protrude in parallel from and perpendicular to the first surface.

In another embodiment, the microinjector chip is manufactured from a biocompatible material selected from the group consisting of metals, polymers, quartz and silica-based materials. In another embodiment, the microinjector chip surfaces and the microinjector chip projections are manufactured from the same material. In another embodiment, the microinjector chip surfaces and said microinjector chip projections are manufactured from different materials.

In another embodiment, the microinjector chip is manufactured by a method selected from the group consisting of lithography, stamping, LIGA, thermoplastic micropattern transfer, resin-based microcasting, micromolding, wet isotropic and anisotropic etching, laser assisted chemical etching, electron etching, and reactive ion etching.

In an embodiment of the present invention, the microinjector chip further comprises an integrated circuit associated with the microinjector chip. In another embodiment, the projections are aligned with the integrated circuit. In another embodiment, the integrated circuit comprises electroconducting material disposed in a bent or branched linear pattern. In yet another embodiment, the integrated circuit comprises electroconducting material disposed in a straight linear pattern. In another embodiment, the integrated circuit induces a piezoelectric effect and causes the projections to vibrate.

In an embodiment of the present invention, the microinjector chip projections are about 25 nm to about 2 μm in diameter. In another embodiment, the projections are about 50 nm in diameter. In another embodiment, the projections are about 1 μm in diameter.

In an embodiment of the present invention, the microinjector chip projections are about 250 nm to about 5 μm long. In another embodiment, the projections are about 500 nm long. In another embodiment, the projections are about 3 μm long.

In another embodiment of the present invention, the injection materials are a purified material or a mixture of materials selected from the group consisting of drugs, peptides, proteins, nucleic acids, polysaccharides, viruses, chromosomes, synthetic particles optionally containing or coated with a macromolecule of interest, spores, plasmids, cell organelles, vesicles, liposomes, micelles, and emulsions. In another embodiment, the injection materials are substantially free of aqueous solutions at the time of injection.

In one embodiment of the present invention, a method of introducing an injection material into a plurality of cells is provided comprising coating the projections of a microinjector chip with an injection material; bringing the projections of the microinjector chip in close proximity to the plurality of cells; piercing the plurality of cells with the projections; and releasing the injection material into the plurality of cells wherein the injection material is substantially free of water at the time of injection.

In an embodiment of the present invention, the injection material is selected from the group consisting of drugs, proteins, nucleic acids, peptides, polysaccharides, viruses, chromosomes, synthetic particles, spores, plasmids, cell organelles, vesicles, liposomes, micelles, and emulsions. In another embodiment, the injection material is coated on a magnetic microbead. In another embodiment, the injection material further comprises a dye.

In another embodiment, coating step is a method selected from the group consisting of freezing, freeze-drying, electrostatic attraction, direct attachment, and biological attachment. In another embodiment, the biological attachment is by the use of biological adhesives or fibronectin.

In another embodiment, the releasing step is induced by vibrating the projections causing the injection material to be released into the plurality of cells. In another embodiment, the vibrating is induced by an integrated circuit disposed on the microinjector chip. In another embodiment, the releasing step is induced by the contact of the injection materials with an aqueous environment present in said cells.

In another embodiment of the present invention, the microinjector chip is manufactured from a biocompatible material selected from the group consisting of metals, polymers, quartz, and silica-based materials.

### BRIEF DESCRIPTION OF THE DRAWINGS

FIG. 1 depicts a side view of a microinjector chip according to one embodiment of the present invention.

FIG. 2 depicts one embodiment of the second surface of a microinjector chip according to the teachings of the present invention.

FIG. 3 depicts one embodiment of the first surface of a microinjector chip according to the teachings of the present invention.

FIG. 4 depicts the second surface of the microinjector chip of FIG. 3.

FIG. 5 depicts another embodiment of the first surface of a microinjector chip according to the teachings of the present invention.

FIG. 6 depicts the second surface of the microinjector chip of FIG. 4.

FIG. 7 depicts one embodiment of microinjector chip projections according to the teachings of the present invention.

FIG. 8 depicts reverse transcriptase-polymerase chain reaction analysis of adult stem cells (HT-33) injected with RNA with the microinjector chip according to the teachings of the present invention. Lane W is a template control containing water only; Lane V is a positive control of embryonic stem cells; Lane 1 contains untreated HT-33 cells; Lane 2 contains HT-33 cells injected by a first microinjector chip; and Lane 3 contains HT-33 cells injected by a second microinjector chip.

### DETAILED DESCRIPTION OF THE INVENTION

The present invention provides a microinjector chip device for the rapid injection of a large number of cells with a target molecule with minimal operator involvement and minimal dilution of the target molecule with aqueous solutions. The microinjector chip device has a first surface and a second surface and a plurality of projections extending from the first surface about perpendicular to the first surface. Injection materials are coated onto the projections allowing the transfer of the substantially liquid-free injection materials into the cells. The microinjector chip device pierces the target cells and the injection material is deposited within the cells. Methods of making the microinjector chip devices, coating them with injection materials and delivering the injection materials to target cells are also provided.

As used herein, “substantially free” refers to injection materials having less than 10% w/v aqueous components.

All of the methods currently available for microinjection of materials into cells have one common drawback. In each of these methods, the injection material is suspended in a carrier liquid, usually an aqueous liquid. The amount of injection material provided to each cell is dependent on the maximum amount of material which can be suspended in the liquid and the maximum volume of liquid that can be introduced into a cell. In order to increase the amount of injection material provided to a cell, it is advantageous to limit the amount of liquid.

The microinjector chip device of the present invention allows injection materials to be coated onto the microinjector chip's projections and delivered to cells without the diluting effects of carrier liquids. The injection material can be coated onto the microinjector chip projections by a variety of methods including, but not limited to, freezing, freeze drying, direct attachment, electrostatic attraction, or through the use of biological adhesives or fibronectin. In one embodiment, the injection material is coated on both the chip body and the projections. In another embodiment, the injection material is coated on only the projections.

In one embodiment of the present invention, the projections of the microinjector chip are magnetized such that injection material-coated magnetic microbeads will attach thereto and, after the microinjector chip projections are brought into contact with and pierce the target cells, the magnetic field is released, the microbeads are released into the cell and the microinjector chip is removed. In another embodiment the microbeads are attracted and attached to the projections via electrostatic attraction or a temperature-associated attraction.

In another embodiment of the present invention, an electrostatic charge is applied to the projections of the microinjector chip and the projections are then dipped into a solution of injection material such that molecules with the solution are attracted to and attach to the projections. The microinjector chip projections are then brought into contact with and pierce the target cells, the electrostatic attraction is removed by grounding the chip, the injection material is released into the cell and the microinjector chip is removed.

In yet another embodiment of the present invention, the microinjector chip projections are dipped into a concentrated solution of injection material in a sample plate and the injection material is freeze-dried onto the projections. The microinjector chip projections are then brought into contact with and pierce the target cells and the injection materials become rehydrated and are released into the cell after a period of time.

The sample plate is preferably coated with a non-stick substance to prevent adherence of the injection material to the plate. Non-stick substances suitable for use on the sample plate are any biocompatible substance including, but not limited to, Teflon® and silicon-based substances. Alternatively, the sample plate can be coated with a bioactive material, such as but not limited to antibodies, hormone or ligands.

Additionally, the sample plate can be used to hold the target cells during the deposition of the injection material.

The injection material can be freeze dried onto the microinjector chip projections through a variety of methods. In one embodiment, the injection material is freeze dried onto the microinjector chip projections by dipping the projections into a concentrated solution of injection material in the sample plate, freezing the microinjector chip and sample plate together, removing the sample plate and drying the injection material onto the projections.

In another embodiment, the projections are coated with fibronectin or a biological adhesive prior to dipping into the concentrated solution of injection material in the sample plate. The injection material is then allowed to adhere to the projections and the injection material injected into the target cells. When fibronectin or biological adhesive are used to attach the injection material to the projections, the projections may need to be left in contact with the target cells for a period of time from several seconds to several days for the injection materials to become disassociated from the projections and be released into the cells. In one embodiment, the biological adhesive is active at temperatures lower than 37° C. and when raised to 37° C., as when the projections enter the target cell, release the injection material into the cells.

In yet another embodiment of the present invention, the projections are supercooled then dipped into a concentrated solution of injection material which then freezes onto the projections. The projections are then warmed slightly and the microinjector chip is brought into contact with and pierces the target cells while warming to 37° C. to allow the injection material to be released into the target cells.

In another embodiment of the present invention, the projections are manufactured from a piezoelectric material and coated with an injection material by any of the foregoing methods. In order to introduce the injection material into the target cells, an electrical field is applied to the microinjector chip causing the projections to change shape or elongate, thereby piercing the cells and depositing the injection material into the target cells.

Additionally, another embodiment provides for manufacturing the projections from a thermally active material that changes shape when heated or cooled. By causing the projections to retract when cooled and lengthen when heated, attached injection materials can be introduced and released into cells.

The injection material is any material that it is desired to inject into the cell. The injection material can be a purified material or a mixture of materials. In particular, the material for injection can be a macromolecule, for example a peptide, protein, nucleic acid or polysaccharide, and analogues and conjugates thereof. Also the injection material may comprise particles, for example viruses, chromosomes, synthetic particles optionally containing or coated with a macromolecule of interest, including, without limitation, spores, plasmids, cell organelles, vesicles, liposomes, micelles and emulsions. Optionally a label, for example a dye, such as a fluorescent label, may be added to the injection material to act as a marker to indicate that the injection is successful. In another embodiment, the injection material is a drug.

In one embodiment of the microinjector chip of the present invention, the microinjector chip projections are coated with an injection material comprising the contents of a particular cell or cell type and then the injection material is introduced into a second cell or cell type. In a non-limiting example the injection material is from an embryonic stem cell and the second cell is a quiescent cell from a spermatogonial stem cell population.

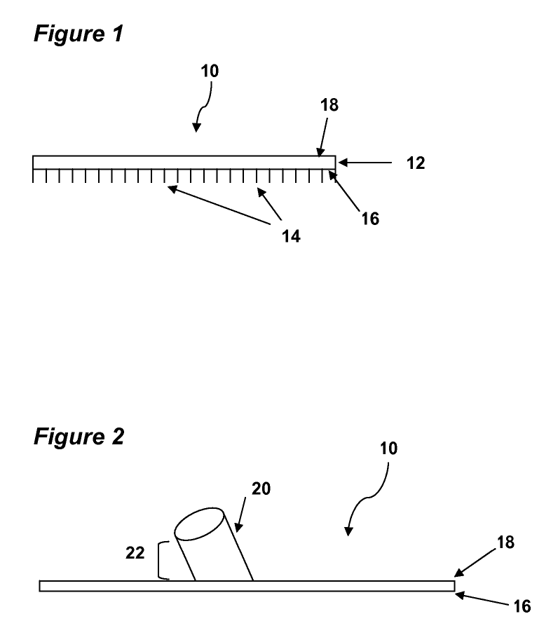

FIG. 1 depicts one embodiment of the microinjector chip of the present invention. The microinjector chip 10 comprises a chip body 12 with a flat surface having a first surface 16 and a second surface 18, and the first surface 16 has a plurality of projections 14 suitable for coating with molecules to be injected into target cells. The projections are solid or hollow substantially rigid structures protruding in roughly one direction from the surface of the chip and do not move significantly with respect to the rest of the chip. However, depending on the manufacturing method and the material from which the chip and projections are fabricated, some movement may occur. The chip may be fabricated in any shape suitable for injecting cells including, but not limited to, round, square and rectangular.

FIG. 2 depicts one embodiment of microinjector chip 10 having a hollow tube 20 protruding at an angle 22 from the second surface 18. Hollow tube 20 is an optional feature of microinjector chip 10. Hollow tube 20 is a coupling facilitator which allows attachment of the microinjector chip 10 to a micromanipulator or microinjection apparatus. Exemplary, non-limiting, micromanipulator and microinjection apparatuses include those manufactured by Eppendorf (Hamburg, Germany) and Narashige (East Meadow, N.Y.). Hollow tube 20 has a diameter of about 50,000 nm to about 100,000 nm, a length of about 100,000 nm to about 200,000 nm and a wall thickness of about 2,500 nm to about 10,000 nm. In embodiments of the present invention, angle 22 is between about 45° and about 85°. In one embodiment, angle 22 is between about 55° and about 65°. In another embodiment, angle 22 is about 65°. Furthermore, hollow tube 20 can optionally be present on microinjector chip 15.

In one embodiment of a microinjector chip of the present invention, the projections are between about 5 nm and about 5 μm in width and between about 10 nm and about 10,000 μm in length. The size and length of the projections are based on the type and size of the target cell and on the type of injection material used. Therefore it is within the scope of the present invention to provide microinjector chips with projections of a variety of sizes to accommodate a variety cell types and injection materials. The projections can be spaced on the surface of the chip in any configuration suitable for the particular target cell. In one embodiment of the microinjector chip of the present invention, the projections are spaced about equidistant from each other and preferably not more than one cell diameter apart from each other. For example, and not intended as a limitation, to deliver injection materials to a culture of target cells having an approximate diameter of 15 μm, the projections are preferably less than about 15 μm apart. FIG. 7 depicts a microinjector chip 10 having projections 14 on the first surface 16 of the microinjector chip wherein the projections 14 are spaced about equidistant from each other.

The microinjector chip projections also generally have a width compatible with the dimension of the cells to be injected. In one embodiment, the width of the projection is between about 1% and about 50% of the cell diameter. In general, cell diameters are from about 10 μm to about 50 μm, however the diameter will vary according to the cell type.

In another embodiment of the present invention, the microinjector chip projections are hollow. The hollow projections define a tube with a first end and a second end wherein the first end is non-releasably attached to the first surface of the microinjector chip and the second end extends from said first surface substantially perpendicular to the surface.

The microinjector chip and projections can be manufactured from a variety of biocompatible metals, polymers or silica-based materials. In one embodiment of the microinjector chip of the present invention, the chip is fabricated from a heat-conducting material. In another embodiment, the chip is fabricated from an electricity-conducting material.

In another embodiment of the microinjector chip, the projections are manufactured from the same material as the body of the chip. In yet another embodiment the body of the chip and the projections are manufactured from different materials.

Suitable techniques for manufacturing the microinjector chips of the present invention include, but are not limited to, lithography, stamping, LIGA (involving lithography, electroplating and molding), thermoplastic micropattern transfer, resin-based microcasting, micromolding in capillaries (MIMIC), wet isotropic and anisotropic etching, laser assisted chemical etching (LACE), vapor deposition, reactive ion etching (RIE), electron etching and other techniques known within the art of chip fabrication.

In another embodiment of the present invention, the microinjector chip is manufactured from quartz and has an integrated circuit placed on the back that aligns with every projection and is used to electronically stimulate the projections to vibrate the injection material off the projections into the cells.

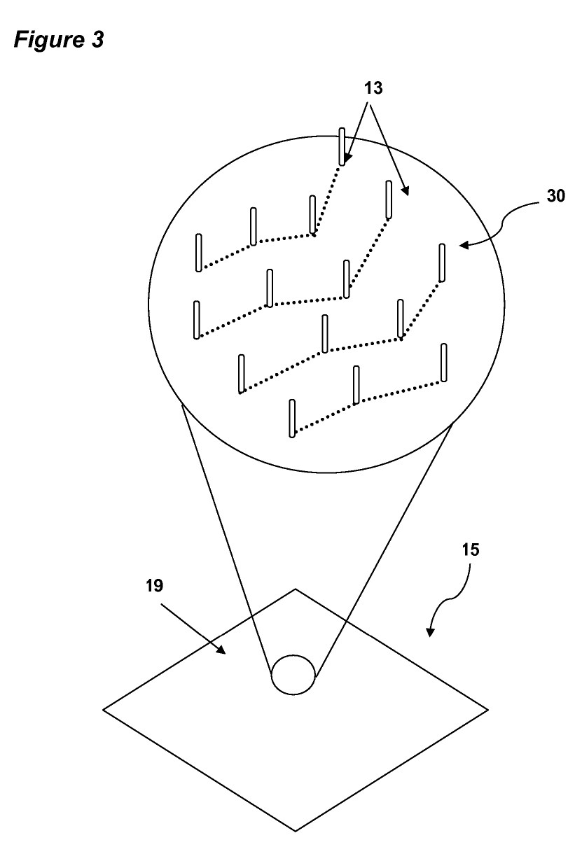

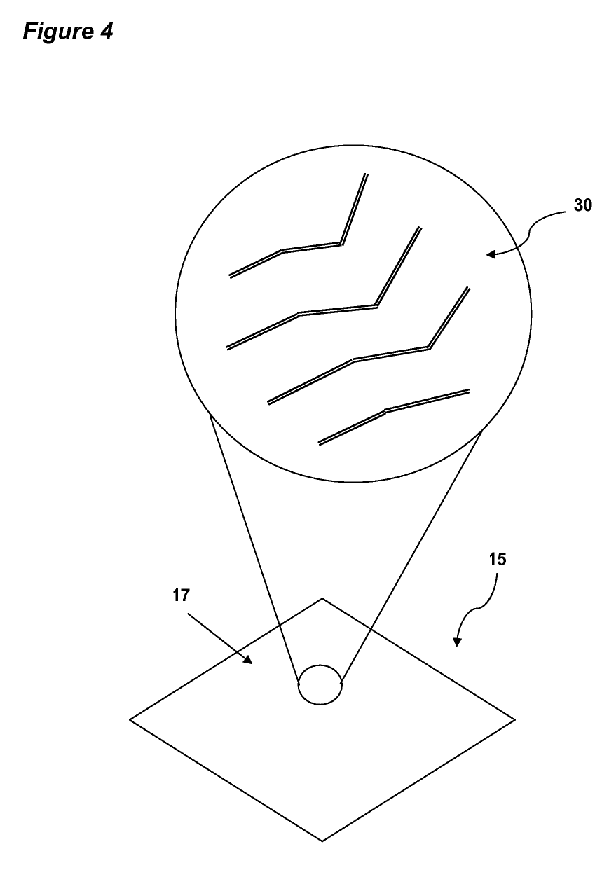

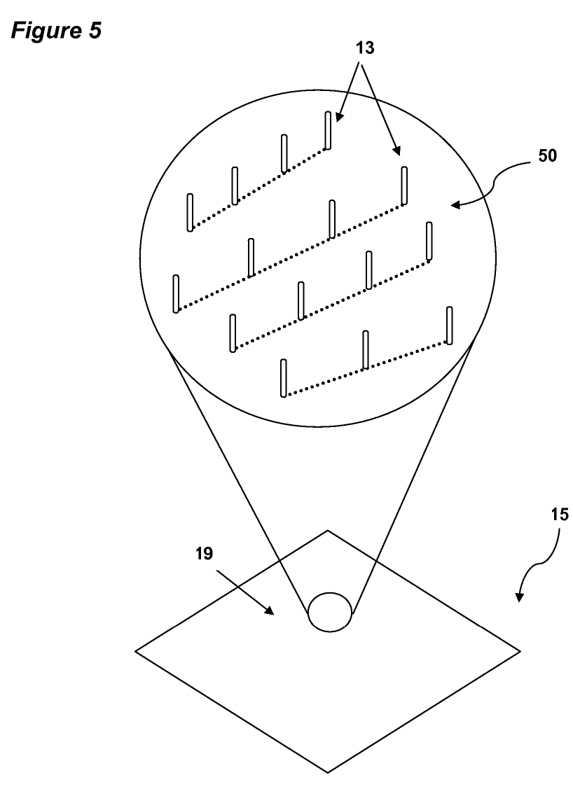

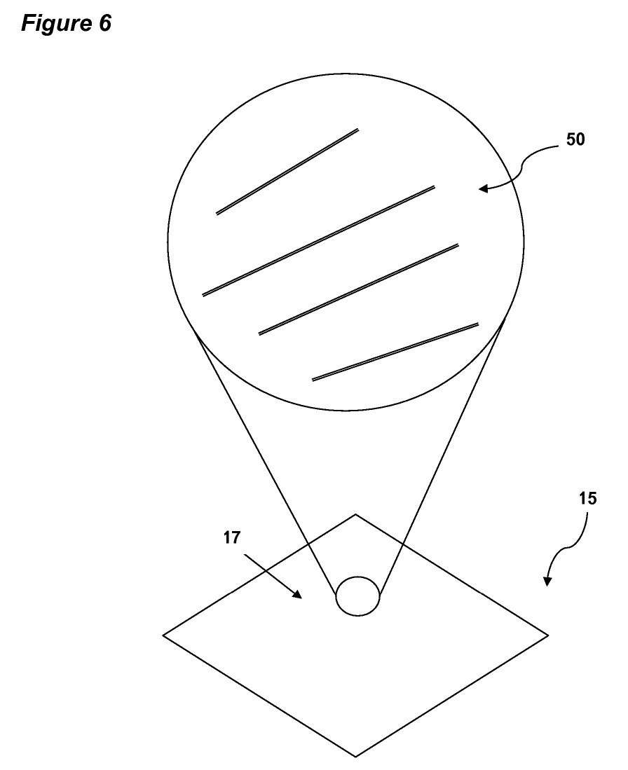

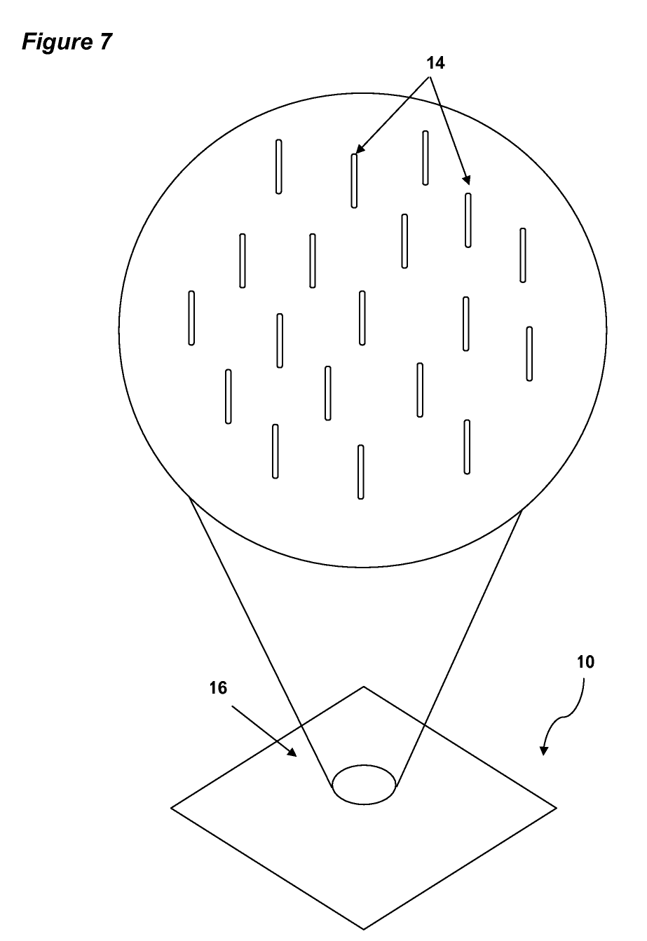

FIGS. 3-6 depict microinjector chips 15 having circuits on the second surface 17 (second surfaces depicted on FIGS. 4 and 6) such that projections 13 on the first surface 19 are aligned with the circuits and conduct electrical signals to the projections (first surfaces depicted on FIGS. 3 and 5). Specifically FIG. 3 depicts a microinjector chip 15 having projections 13 aligned with a circuit 30 comprising electroconducting material disposed in a bent or branched linear pattern on the second surface 17. FIG. 4 depicts the second surface 17 of the same microinjector chip as FIG. 3 depicting the electroconducting circuit 30 disposed on the second surface 17.

FIG. 5 depicts an alternative embodiment of microinjector chip 15 wherein projections 13 are aligned with a circuit 50 comprising electroconducting material disposed in a straight linear pattern on second surface 17 and projections 13 are aligned with and extend perpendicular from circuit 50. FIG. 6 depicts the second surface 17 of the same microinjector chip as FIG. 5 depicting the electroconducting circuit 50 disposed on the second surface 17.

In another embodiment of the present invention, an integrated circuit is present at the base of each projection on the same side of the chip as the projections. This style of microinjector can produce a piezoelectric effect to vibrate the injection material off the projection. Alternatively, a thin film deposition of an electroconducting material can be placed on the projection side of the chip and/or on the projections, which can then be electrified to create a piezoelectric effect.

These circuits may be placed on any material that has a piezoelectric effect, i.e. the chips can be made out of any suitable material to achieve similar results to a quartz chip (i.e. lithium niobate, zinc oxide, etc.).

### EXAMPLE 1

### Injection of RNA into Cells Using a Microiniector Chip

HT33 cells (adult human male derived gonadal stem cells) were plated 24 hours prior to injections with microinjector chip to allow for attachment.

In vitro transcription of mRNAs encoding human Oct-4, Sox2, c-Myc, and KLf4 was done using T7 and SP6 AmpliScribe Kits (followed manufacturer's recommended procedure, Epicentre Biotechnologies, Madison, Wis.). Linearized human Oct-4, Sox2, c-Myc, and KLf4 constructs were used as the template for RNA transcription and were obtained from Open Biosystems (Huntsville, Ala.). Approximately 65 μg of RNA for each transcribed sequence was pooled together to yield a total amount of RNA of approximately 260 μg in a total volume of 100 μl; a concentration of RNA at 2.6 μg/μl. One μl drops were applied onto the microinjector chip such that each biochip had 50 “drops” arranged randomly. Two chips were prepared in this manner. The chips were then incubated for approximately 30 minutes in a lyophilizing machine to freeze-dry the RNA to the chip thereby coating the chip projections with the RNA.

Medium was removed from the plates containing the attached HT33 cells. The coated chips were then inverted and the microinjector projections placed directly onto the cells. The cells were then incubated for approximately 5 minutes with the RNA-containing side of the chips. Two plates of cells were each exposed to different chips. An additional plate was used for a negative control, in which the cells were not exposed to a microinjector chip. Following the chip incubation period, the plates were washed twice with medium to remove any detached cells, and cells were incubated under standard culturing conditions. Five days after transfer of RNA from the microinjector chip to the HT-33 cells, the cells were harvested for reverse transcriptase polymerase chain reaction (RT-PCR) analysis.

The injected HT-33 cells were harvested by rinsing the plates with PBS and/or scraping plates with a cell scraper. RNA was isolated using the RNeasy Mini Kit (Qiagen, Valencia, Calif.). Approximately 2 μg of total RNA was Dnase I (Invitrogen, Carlsbad, Calif.) treated, and the treated RNA was used for cDNA synthesis using Omniscript reverse transcriptase (Qiagen). Approximately 25 ng of cDNA was then used for each RT-PCR reaction. RT-PCR was carried out using HotStarTaq Plus DNA Polymerase (Qiagen).

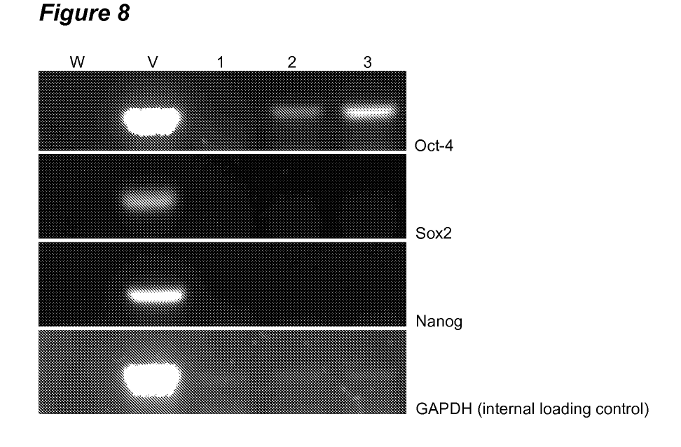

As can be seen in FIG. 8, the Oct-4 RNA was only been detected in cells that were injected with RNA using the microinjector chip. Therefore, delivery of RNA that has been coated on the microinjector chip projections can be successfully introduced into the cell.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on those embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are individually incorporated by reference herein in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

I claim:

1. A microinjector chip for delivery of injection materials to a plurality of cells comprising:
a microinjector chip having a first surface and a second surface; and
a plurality of projections protruding from said first surface wherein said injection materials are coated onto at least a subset of said projections.

2. The microinjector chip of claim 1 wherein said plurality of projections protrude in parallel from and perpendicular to said first surface.

3. The microinjector chip of claim 1 wherein said microinjector chip is manufactured from a biocompatible material selected from the group consisting of metals, polymers, quartz and silica-based materials.

4. The microinjector chip of claim 1 wherein the microinjector chip surfaces and said microinjector chip projections are manufactured from the same material.

5. The microinjector chip of claim 1 wherein said microinjector chip surfaces and said microinjector chip projections are manufactured from different materials.

6. The microinjection chip of claim 1 wherein said microinjector chip is manufactured by a method selected from the group consisting of lithography, stamping, LIGA, thermoplastic micropattern transfer, resin-based microcasting, micromolding, wet isotropic and anisotropic etching, laser assisted chemical etching, electron etching, and reactive ion etching.

7. The microinjector chip of claim 1 further comprising an integrated circuit associated with said microinjector chip.

8. The microinjector chip of claim 7 wherein said projections are aligned with said integrated circuit.

9. The microinjector chip of claim 7 wherein said integrated circuit comprises electroconducting material disposed in a bent or branched linear pattern.

10. The microinjector chip of claim 7 wherein said integrated circuit comprises electroconducting material disposed in a straight linear pattern.

11. The microinjector chip of claim 6 wherein said integrated circuit induces a piezoelectric effect and causes said projections to vibrate.

12. The microinjector chip of claim 1 wherein said projections are about 25 nm to about 2 μm in diameter.

13. The microinjector chip of claim 12 wherein said projections are about 50 nm in diameter.

14. The microinjector chip of claim 12 wherein said projections are about 1 μm in diameter.

15. The microinjector chip of claim 1 wherein said projections are about 250 nm to about 5 μm long.

16. The microinjector chip of claim 15 wherein said projections are about 500 nm long.

17. The microinjector chip of claim 15 wherein said projections are about 3 μm long.

18. The microinjector chip of claim 1 wherein said injection materials are selected from the group consisting of a purified material or a mixture of materials.

19. The microinjector chip of claim 1 wherein said injection material is selected from the group consisting of drugs, peptides, proteins, nucleic acids, polysaccharides, viruses, chromosomes, synthetic particles optionally containing or coated with a macromolecule of interest, spores, plasmids, cell organelles, vesicles, liposomes, micelles, and emulsions.

20. The microinjector chip of claim 1 wherein said injection materials are substantially free of aqueous solutions at the time of injection.

21. A method of introducing an injection material into a plurality of cells comprising;
coating the projections of a microinjector chip with an injection material;
bringing said projections of said microinjector chip in close proximity to said plurality of cells;
piercing said plurality of cells with said projections; and
releasing said injection material into said plurality of cells wherein said injection material is substantially free of water at the time of injection.

22. The method according to claim 21 wherein said injection material is selected from the group consisting of drugs, proteins, nucleic acids, peptides, polysaccharides, viruses, chromosomes, synthetic particles, spores, plasmids, cell organelles, vesicles, liposomes, micelles, and emulsions.

23. The method according to claim 21 wherein said injection material is coated on a magnetic microbead.

24. The method according to claim 21 wherein said injection material further comprises a dye.

25. The method according to claim 21 wherein said coating step is a method selected from the group consisting of freezing, freeze-drying, electrostatic attraction, direct attachment, and biological attachment.

26. The method according to claim 25 wherein said biological attachment is by the use of biological adhesives or fibronectin.

27. The method according to claim 21 wherein said releasing step is induced by vibrating said projections causing the injection material to be released into said plurality of cells.

28. The method according to claim 27 wherein said vibrating is induced by an integrated circuit disposed on said microinjector chip.

29. The method according to claim 21 wherein said releasing step is induced by the contact of said injection materials with an aqueous environment present in said cells.

30. The microinjector chip of claim 21 wherein said microinjector chip is manufactured from a biocompatible material selected from the group consisting of metals, polymers, quartz, and silica-based materials.
